(19) United States
(12) Patent Application Publication
(10) Pub. No.: US 2007/0219541 A1
(43) Pub. Date: Sep. 20, 2007

(54) System and method for ophthalmic laser surgery on a cornea

(75) Inventor: Ronald M. Kurtz, Irvine, CA (US)

Correspondence Address: CONNOLLY BOVE LODGE & HUTZ LLP, P.O. BOX 2207, WILMINGTON, DE, 19899, US

(73) Assignee: INTRALASE CORP.

(21) Appl. No.: 11/375,542

(22) Filed: Mar. 14, 2006

(51) Int. Cl. A61B 18/18

(52) U.S. Cl. 606/4

(57) ABSTRACT

A system and method for ophthalmic laser surgery is disclosed. An optical scanner is employed to create a thickness profile for the cornea. A lens is placed in contact with the anterior surface of the cornea. The thickness profile is then used to identify a scan region within the cornea, wherein the scan region is disposed at approximately a uniform distance from the posterior surface of the cornea. A focal point of a laser beam is thereafter scanned along a path within the scan region to create an intracorneal incision.

### BACKGROUND OF THE INVENTION

1. Field of the Invention

The field of the present invention is ophthalmic surgical systems and methods, and particularly systems and methods for surgically incising corneal tissue.

2. Background

Presently, ophthalmic surgical systems and techniques focus on making an incision within the cornea such that the incision is disposed at a constant distance from the anterior surface of the cornea. For example, each of U.S. Pat. No. 5,993,438, U.S. Pat. No. 6,730,074, and U.S. Patent Application Publication No. 20050245915 teaches that making an intracorneal incision at a uniform distance from the anterior surface of the cornea is desirable. However, for a patient whose cornea has an irregular thickness profile, such incisions fail to address the irregularities during the surgical procedure when steps may be easily taken to correct the irregularities.

### SUMMARY OF THE INVENTION

The present invention is directed towards a system and method for ophthalmic laser surgery. In performing the surgical procedure, a thickness profile of the cornea is generated, preferably using an optical coherence tomography scanner. A contact lens is placed against the anterior surface of the cornea. The posterior surface of the lens, i.e., that surface which is placed in contact with the cornea, may be curved or planar, with the lens being adapted to conform the shape of the anterior surface of the cornea to the curvature of the lens. The thickness profile is thereafter used to identify a scan region within the cornea. The scan region is disposed at approximately a uniform distance from the posterior surface of the cornea and preferably has a geometric shape which is best-fit to a plurality of points identified within the cornea, each of which is equidistant from the posterior surface of the cornea. Once the scan region has been identified, a laser scanner is employed to scan the focal point of a laser beam along a path within the scan region.

Accordingly, an improved a system and method for ophthalmic laser surgery are disclosed. Other objects and advantages will appear hereinafter.

### BRIEF DESCRIPTION OF THE DRAWINGS

In the drawings, wherein like reference numerals refer to similar components:

FIG. 1 is a sectional view of a cornea showing a corneal incision at a fixed distance from the anterior surface of the cornea according to the prior art;

FIG. 2 is a sectional view of a cornea showing a corneal incision at a fixed distance from the posterior surface of the cornea;

FIG. 3A is a sectional view of a cornea showing a scan region defined within the cornea as preparation for an incision;

FIG. 3B illustrates sampling locations about the cornea used for creating a thickness profile for the cornea; and

FIG. 4 schematically illustrates a system for performing ophthalmic laser surgery.

### DETAILED DESCRIPTION OF THE PREFERRED EMBODIMENTS

Turning in detail to the drawings, FIG. 1 illustrates an example of a corneal incision made using the teachings of the prior art. The cornea 10, for purposes of this example, has an irregular thickness profile, i.e. the thickness of the cornea 10 is ‘a’ at a first location and is ‘b’ at a second location, where the thicknesses ‘a’ and ‘b’ are unequal. For purposes of this disclosure, an irregular thickness profile of a cornea is defined as any cornea that does not have a regular, constant thickness across a substantial portion of the cornea. Thus, the intracorneal incision 12, when made in accordance with the practices and teachings of the prior art, is formed at a constant distance, ‘c’, from the anterior surface 14 of the cornea. Because the cornea 10 has an irregular thickness profile, the distance of the intracorneal incision 12 from the posterior surface 16 of the cornea 10 varies. In this example, the intracorneal incision 12 is at a distance ‘d’ from the posterior surface 16 of the cornea 10 at a first location and at a distance ‘e’ from the posterior surface 16 of the cornea 10 at a second location, where the distances ‘d’ and ‘e’ are unequal.

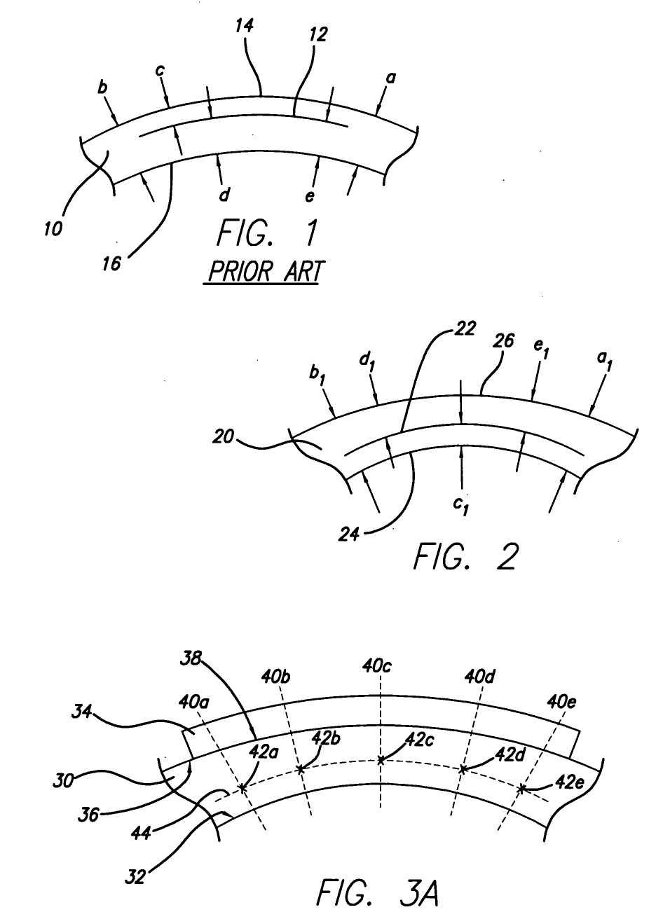

FIG. 2 illustrates an example of a corneal incision made using the methods and systems of the present invention, both of which are described in further detail below. The cornea 20 of this example is also characterized by an irregular thickness profile, although the procedure could be performed on a cornea having a uniform thickness. The cornea 20 has a first thickness ‘a₁’ at a first location and a second thickness ‘b1’ at a second location, where the thicknesses ‘a₁’ and ‘b₁’ are unequal. Unlike the prior art, however, the intracorneal incision 22 is formed at a substantially uniform distance, ‘c₁’, from the posterior surface 24 of the cornea. In this example, because the cornea 20 has an irregular thickness profile, the intracorneal incision 22 is at a distance ‘d1’ from the anterior surface 26 of the cornea 10 at a first location and at a distance ‘e1’ from the anterior surface 26 of the cornea 10 at a second location, where the distances ‘d₁’ and ‘e₁’ are unequal. Once the intracorneal incision 22 is made, additional incisions may be made to form a flap in the anterior surface of the cornea, to excise the part of the cornea between the anterior surface and the intracorneal incision 22, or for any other appropriate purpose.

Forming the intracorneal incision at a substantially uniform distance from the posterior surface of the cornea may be beneficial for many different types of ophthalmic laser surgery. For example, such an incision may be used when harvesting tissue from the corneal endothelium. Having the incision disposed at a substantially uniform distance from the posterior surface of the cornea means that the harvested tissue has a uniform thickness and ensures that any irregularities in the thickness of the donor's cornea are not reproduced when the tissue is grafted onto a recipient's cornea.

By way of another example, such an incision may be made in a recipient's cornea for purposes of a partial corneal graft. For such a graft, the corneal tissue excised from the donor cornea is preferably removed using the methods taught in the prior art as described above, i.e. the excised tissue is removed by making an incision which is at a uniform distance from the anterior surface of the cornea. Thus, when the donor tissue is grafted onto the recipient's cornea, any irregularities in thickness which were present in either the recipient's cornea or in the donor cornea eliminated by virtue of the placement of the incisions within both corneas, and the cornea resulting from the graft will therefore have a uniform thickness.

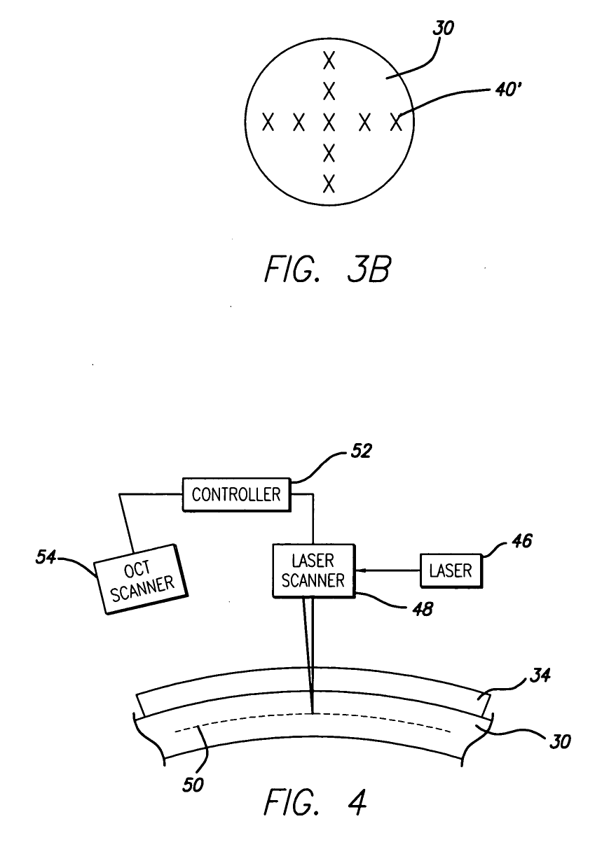

FIG. 3A shows, by way of illustration, the process for determining the location of the incision within the cornea 30 so that the incision is at a substantially uniform distance from the posterior surface 32 of the cornea 30. Initially, a thickness profile of the cornea 30 is created, although this step could also be performed after placement of the lens 34. This thickness profile may be generated by any one of the many known methods for measuring the physical structure of the eye. The preferred method of generating the thickness profile is through optical coherence tomography, which has many different variations known in the art. Many commercially available OCT scanners are capable of generating the thickness profile. One example is the Visante™ OCT scanning system, manufactured by Carl Zeiss Meditec, which has an office in Dublin, Calif. One advantage of the Visante™ OCT system is that it does not make contact with the eye when performing the OCT scan. The thickness profile may be created through a whole scan of the cornea 30, or it may be created through a scan at select locations 40a-e around the cornea 30. FIG. 3B illustrates an example of how the sample locations 40′ may be spread about the cornea 30 for purposes of developing the thickness profile.

The lens 34 is placed in contact with the anterior surface 36 of the cornea 30. The physical dimensions of the lens 34 are known in advance and are used in the surgical procedure to help properly position the focal point of the laser. The posterior surface 38 of the lens 34 has a curvature, and the lens 34 is formed of a material that is sufficiently rigid such that the anterior surface 36 of the cornea conforms 30 to the curvature of the posterior surface 38 of the lens 34. Such lenses and the associated support structure are well known in the art. In practice, the curvature of the posterior surface 38 of the lens 34 may be planar or radially defined, or may have any other appropriate geometric form.

With the lens 34 in place, for each of the select locations 40a-e, points 42a-e are identified within the cornea 30 using the posterior surface 38 of the lens 34 as a reference. Each of the points 42a-e is disposed at a predetermined distance from the posterior surface 32 of the cornea 30. A curved surface 44 is then best-fit to the points 42a-e. The actual shape of the curved surface 44 may be almost any shape, but it will generally depend upon factors such as the thickness profile of the cornea and the curvature of the posterior surface of the lens 34. If the lens 34 is an applanation lens, then the resulting curved surface could be planar. Similarly, if the curvature of the lens 34 is radially defined, then the resulting curved surface may also be radially defined, or it may be defined by multiple radii such that it is elliptical in overall shape.

By forming the curved surface 44 in this manner, it is at a substantially uniform distance from the posterior surface 32 of the cornea 30. This process, however, does not take into account variances in the corneal thickness which are small and localized. Therefore, the curved surface 44, near these small, localized variances, may be closer or further away from the posterior surface 32 of the cornea 30.

The portion of the curved surface 44 included within the operable scan range of the laser scanner (see FIG. 4) is the scan region for the ensuing surgical incision. The entire scan region may be utilized for the surgical incision, or optionally, the incision may be limited to a sub-section of the scan region. Regardless of scan region utilization, however, the incision is made in accordance with known practices by scanning the focal point of the laser beam along one or more selected paths within the scan region.

FIG. 4 schematically illustrates a system for performing the incision described above. With the lens 34 positioned in place on the cornea 30, light from the laser 46 is directed into the cornea 30 by the laser scanner 48 such that the focal point of the laser beam is within the scan region 50. The laser 46 may be of the type described in U.S. Pat. No. 4,764,930 and preferably produces an ultra-short pulsed beam as described in U.S. Pat. No. 5,984,916, the disclosures of which are incorporated herein by reference. The laser scanner 48 is preferably of the type disclosed in copending U.S. patent application Ser. No. 11/272,571, the disclosure of which is incorporated herein by reference. The controller 52 electronically controls the laser scanner 48 to scan the focal point of the laser beam and make the desired incision. The optical coherence tomography (OCT) scanner 54 may also be electronically controlled by the controller 52, or they may be embodied in separate systems wherein data generated by the OCT scanner 54 is transferred to the controller 52 for use during the surgical procedure. As indicated above, the OCT scanner 54 is preferably employed prior to placement of the lens 34 to generate the corneal thickness profile.

During operation, the controller 52 uses the information relating to the thickness of the cornea that was generated by the OCT scanner 54. From this information, the controller 52 constructs the thickness profile, identifies the scan region once the lens 34 is in place, and controls the laser scanner to scan the focal point within the scan region, thereby making the desired incision. User interaction with the controller 52 during this process is optional.

Thus, a system and method for ophthalmic laser surgery are disclosed. While embodiments of this invention have been shown and described, it will be apparent to those skilled in the art that many more modifications are possible without departing from the inventive concepts herein. The invention, therefore, is not to be restricted except in the spirit of the following claims.

What is claimed is:

1. A method for incising a cornea, the method comprising:
creating a thickness profile of the cornea;
placing a lens in contact with a corneal anterior surface;
identifying a scan region within the cornea using the thickness profile, the scan region being disposed at approximately a uniform distance from a corneal posterior surface; and
scanning a focal point of a laser beam along a path within the scan region.

2. The method of claim 1, wherein placing the lens in contact with the corneal anterior surface includes placing a lens posterior surface in contact with the corneal anterior surface, the lens posterior surface having a curvature and being adapted to conform the shape of the corneal anterior surface to the curvature.

3. The method of claim 2, wherein the curvature of the lens posterior surface is radially defined.

4. The method of claim 1, wherein creating the thickness profile of the cornea includes measuring a thickness of the cornea at a plurality of locations about the cornea.

5. The method of claim 1, wherein identifying the scan region includes identifying a plurality of points within the cornea, the points being disposed equidistant from the corneal posterior surface, and fitting a curved surface to the plurality of points.

6. The method of claim 5, wherein the curved surface is radially defined.

7. The method of claim 5, wherein the curved surface is defined by multiple radii.

8. The method of claim 1, wherein the laser beam comprises a pulsed laser beam.

9. A method for incising a cornea, the method comprising:
measuring the thickness of the cornea at a plurality of locations about the cornea;
placing a lens in contact with a corneal anterior surface;
identifying at each of the locations a plurality of points, each point being disposed equidistant from a corneal posterior surface;
fitting a curved surface to the plurality of points, the curved surface defining a scan region; and
scanning a focal point of a laser beam along a path within the scan region.

10. The method of claim 9, wherein placing the lens in contact with the corneal anterior surface includes placing a lens posterior surface in contact with the corneal anterior surface, the lens posterior surface having a curvature and being adapted to conform the shape of the corneal anterior surface to the curvature.

11. The method of claim 10, wherein the curvature of the lens posterior surface is radially defined.

12. The method of claim 9, wherein the curved surface is radially defined.

13. The method of claim 9, wherein the curved surface is defined by multiple radii.

14. The method of claim 9, wherein the laser beam comprises a pulsed laser beam.

15. An ophthalmic surgical system comprising:
a contact lens having a posterior surface engagable with an anterior surface of a cornea;
a laser source capable of generating a laser beam;
a laser scanner adapted to direct a focal point of the laser beam to a location within the cornea;
an optical coherence tomography scanner adapted to generate a thickness profile of the cornea;
a laser scanner controller adapted to identify a scan region within the cornea based upon the thickness profile, the scan region being disposed at approximately a uniform distance from a corneal posterior surface, and to scan the focal point along a path within the scan region using the laser scanner.

16. The system of claim 15, wherein the lens posterior surface has a curvature, and the lens is adapted to conform the shape of the corneal anterior surface to the curvature.

17. The system of claim 16, wherein the curvature of the lens posterior surface is radially defined.

18. The system of claim 15, wherein the thickness profile comprises a plurality of thickness measurements at various locations about the cornea.

19. The system of claim 15, wherein the scan region comprises a curved surface fit to a plurality of points within the cornea, the points being disposed equidistant from the corneal posterior surface.

20. The system of claim 19, wherein the curved surface is radially defined.

21. The system of claim 19, wherein the curved surface is defined by multiple radii.

22. The system of claim 15, wherein the laser beam comprises a pulsed laser beam.

23. An ophthalmic surgical system comprising:
a contact lens having a posterior surface engagable with an anterior surface of a cornea;
a laser source capable of generating a laser beam;
a laser scanner adapted to direct a focal point of the laser beam to a location within the cornea;
an optical coherence tomography scanner adapted to perform a plurality of thickness measurements at various locations about the cornea;
a laser scanner controller adapted to fit a curved surface to a plurality of points within the cornea, each point being disposed at one of the locations of the thickness measurements and being equidistant from the corneal posterior surface, and to scan the focal point along a path on the curved surface.

24. The system of claim 23, wherein the lens posterior surface has a curvature, and the lens is adapted to conform the shape of the corneal anterior surface to the curvature.

25. The system of claim 24, wherein the curvature of the lens posterior surface is radially defined.

26. The system of claim 23, wherein the curved surface is radially defined.

27. The system of claim 23, wherein the curved surface is defined by multiple radii.

28. The system of claim 23, wherein the laser beam comprises a pulsed laser beam.
